(19) United States
(12) Patent Application Publication
(10) Pub. No.: US 2015/0328198 A1
(43) Pub. Date: Nov. 19, 2015

(54) METHODS OF TREATING METHICILLIN-RESISTANT STAPHYLOCOCCUS AUREUS (MRSA) USING PPAR-GAMMA AGONISTS

(71) Applicant: The University of North Carolina at Chapel Hill, Chapel Hill, NC (US)

(72) Inventors: Anthony Richardson, Chapel Hill, NC (US); Lance Thurlow, Chapel Hill, NC (US)

(21) Appl. No.: 14/713,345

(22) Filed: May 15, 2015

Related U.S. Application Data
(60) Provisional application No. 61/994,577, filed on May 16, 2014.

(51) Int. Cl. A61K 31/4439

(52) U.S. Cl. CPC A61K 31/4439

(57) ABSTRACT

The present invention relates to methods of preventing or treating a staphylococcus infection comprising administering an effective amount of a peroxisome proliferator-activated receptor (PPAR)-γ agonist. The invention further relates to methods of modulating a host wound response.

### STATEMENT OF PRIORITY

This application claims priority to U.S. Provisional Application Ser. No. 61/994,577, filed May 16, 2014, the disclosure of which is incorporated herein by reference in its entirety.

### STATEMENT OF FEDERAL SUPPORT

This invention was made with government support under Grant Nos. AI093613 and AI111707 awarded by the National Institutes of Health. The government has certain rights in the invention.

### FIELD OF THE INVENTION

The present invention relates to methods of treating a Staphylococcus infection using peroxisome proliferator-activated receptor (PPAR)-γ agonists.

### BACKGROUND OF THE INVENTION

Skin and soft tissues infections (SSTIs) caused by Staphylococcus aureus are a major health care burden. Not only can SSTIs disseminate resulting in more severe disease manifestations such as osteomyelitis, sepsis and endocarditis, but the frequency of S. aureus SSTIs is ever increasing as hypervirulent clones become prevalent in the community (e.g. USA300, Thurlow et al. (2012) FEMS Immunol. Med. Microbiol. 65, 5-22). The host response to an S. aureus SSTI is very similar to that of any typical wound. First, there is a robust inflammatory response aimed at sterilizing the damaged tissue (FIG. 1). This response is characterized by an abundance of highly inflammatory, classically activated macrophages (M1-MΦs) and neutrophils (PMNs). These immune cells produce a myriad of inflammatory effectors such as antimicrobial peptides and immune radicals (e.g. superoxide and nitric oxide (NO.)). NO. is enzymatically generated by the inducible NO. synthase or iNOS and thus iNOS expression is a hallmark of classically activated M1-MΦs. Most bacterial challenges encountered by a host are effectively contained by this pro-inflammatory immune response mediated by M1-MΦs and infiltrating PMNs. Once the infection is resolved, the host response shifts into a wound healing resolution phase, during which, inflammation is quelled and cellular proliferation begins as well as the synthesis of extracellular matrix components and dermal fatty acids. This resolving environment requires the actions of alternatively activated macrophages (M2-MΦs) and is essential for the restoration of normal homeostasis in the skin (FIG. 1). M2-MΦs lack iNOS expression, but rather produce Arginase-1 (Arg-1) the committed step to the synthesis of polyamines, which exert anti-inflammatory and cell proliferative effects on tissue (FIG. 1, Panel A). Furthermore, Arg-1 catalyzes the conversion of arginine to ornithine, a precursor to proline. Given that collagen synthesis is heavily reliant on available proline, the induction of Arg-1 in M2-MΦs likely contributes to the observed collagen deposition during the resolution phase. The combined actions of the inflammatory and resolution phases sterilize the wound and return normal tissue homeostasis.

During an S. aureus SSTI, the host response generally follows the above-described progression over a two-week period before tissue homeostasis is restored. Initially, massive inflammation leads to an open lesion that is eventually covered by a scab followed by re-epithelialization (FIG. 1, Panel C). Inflammatory cells encountered during the first week are highly activated PMNs and M1-MΦs expressing iNOS (FIG. 1, Panel D). Eventually, the wound begins to resolve as M2-MΦs arrive and respond to damaged host tissue (FIG. 1, Panel E). After about 1 week, the numbers of M1 -MΦs and M2-MΦs are approximately equal with the transition to predominantly M2-MΦs occurring over the second week of infection (FIG. 1, Panel F). S. aureus is uniquely resistant to many host inflammatory effectors and therefore is not efficiently cleared by the initial host inflammatory phase. This is in direct contrast to other skin pathogens (e.g. S. epidermidis and E. faecalis), which are rapidly cleared by the host inflammatory response (FIG. 1, Panel B). On the other hand, we have demonstrated that the conversion to the M2-MΦ dominant resolution phase is essential to resolve S. aureus SSTIs (Thurlow et al. (2013) Cell Host Microbe 13, 100-107). Part of the protective mechanism of the M2 phase is the redirection of host arginine away from iNOS (S. aureus is highly resistant to NO.) and towards the production of polyamines, compounds that are uniquely toxic to S. aureus. Interestingly, the recent emergence of USA300, the dominant SSTI causing S. aureus strain, is partly due to its acquisition of polyamine-resistance through the activity of a recently characterized polyamine acetyltransferase, SpeG (Thurlow et al. (2013) Cell Host Microbe 13, 100-107; Joshi et al. (2011) Mol. Microbiol. 82, 9-20). During the M2-phase, polyamines kill roughly 92% of the infecting S. aureus, however, USA300 is completely immune to these compounds and is therefore able to persist longer to facilitate transmission to new hosts as well as dissemination to deeper tissue. However, it should be noted that polyamine-resistant USA300 is still effectively cleared during the M2-phase, albeit less than other polyamine-sensitive strains (FIG. 1, Panel B). Therefore, other factors present in host tissue undergoing the wound-resolution program are effective at clearing S. aureus.

Peroxisome Proliferator Activator Receptor-γ (PPAR-γ) is a host regulatory protein that is essential for the gene expression associated with M2-MΦs and wound repair. It activates Arg-1 and other polyamine metabolism genes (e.g. Spermine/Spermidine Acetyl Transferase, S SAT) as well as fatty acid production. Particularly, PPAR-γ induces the synthesis of mono-unsaturated fatty acids (MUFAs) known to be toxic to S. aureus. MUFAs are generated through the activity of Stearol-CoA Destaturase (SCD-1), a gene directly activated by PPAR-γ. SCD-1 deficient mice are known to be hypersusceptible to S. aureus skin infections, though it has not been shown that this is due to the production of MUFAs (Georgel et al. (2005) Infect. Immun. 73, 4512-4521). However, it has long been appreciated that the lipid fraction of healing wounds exerts strong anti-staphylococcal activity (Heczko et al. (1979) J. Clin. Microbiol. 9(3): 333-335).

### SUMMARY

A first aspect of the invention is a method of preventing or treating a Staphylococcus infection comprising administering an effective amount of a peroxisome proliferator-activated receptor (PPAR)-γ agonist to a subject in need thereof.

In some aspects, the Staphylococcus infection is present on the skin. In further aspects, the staphylococcus infection is present in a wound.

In particular aspects, the subject does not have diabetes and/or is not being treated for diabetes with a PPAR-γ agonist of the present invention.

According to some aspects, the PPAR-γ agonist is administered orally, parenterally, by inhalation spray, topically, transdermally, rectally, nasally, sublingually, buccally, vaginally or via an implanted reservoir.

Aspects of the present invention further provide methods of modulating a host wound response comprising administering an effective amount of a PPAR-γ agonist to the host in an amount to elevate production of polyamines and/or mono-unsaturated fatty acids compared to the levels present in the absence of administration of a PPAR-γ agonist.

In some aspects, an increase in the level of production of polyamines and/or mono-unsaturated fatty acids indicates repair of skin tissue.

In further aspects, the wound response is initiated by methicillin-resistant Staphylococcus aureus (MRSA).

In still further aspects, the PPAR-γ agonist is a thiazolidinedione. These and other aspects of the invention are set forth in more detail in the description of the invention below.

### BRIEF DESCRIPTION OF THE DRAWINGS

Having thus described the presently disclosed subject matter in general terms, reference will now be made to the accompanying Drawings, which are not necessarily drawn to scale.

FIG. 1. The wound healing response coincides with rapid S. aureus clearance. Panel A. Fates of host arginine during wound healing. Pro-inflammatory cytokines skew M1-MΦs into producing iNOS and generating NO.. Anti-inflammatory cytokines (IL-4 and IL-13) induce M2-MΦs to produce polyamines and proline for collagen synthesis. Panel B. Viable bacteria within a murine SSTI. Unlike other pathogens, S. aureus remains fully viable for the first week, but then begins to clear. Here clearance of polyamine-resistant USA300 is demonstrated. Traditional MRSA clones are killed even faster during the second week of infection Panel C. H&E staining of S. aureus SSTI as it progresses through both phases of the host response. Panel D. iNOS staining indicates that M1-MΦs dominate early and are scarce at the end of the infection. Blue is DAPI counterstain Panel E. In contrast, Arg-1 staining reveals that M2-MΦs begin to arrive at day 7 and dominate the response late in infection. Panel F. Flow cytometric quantification of the M2-MΦ takeover late in infection when S. aureus is rapidly cleared.

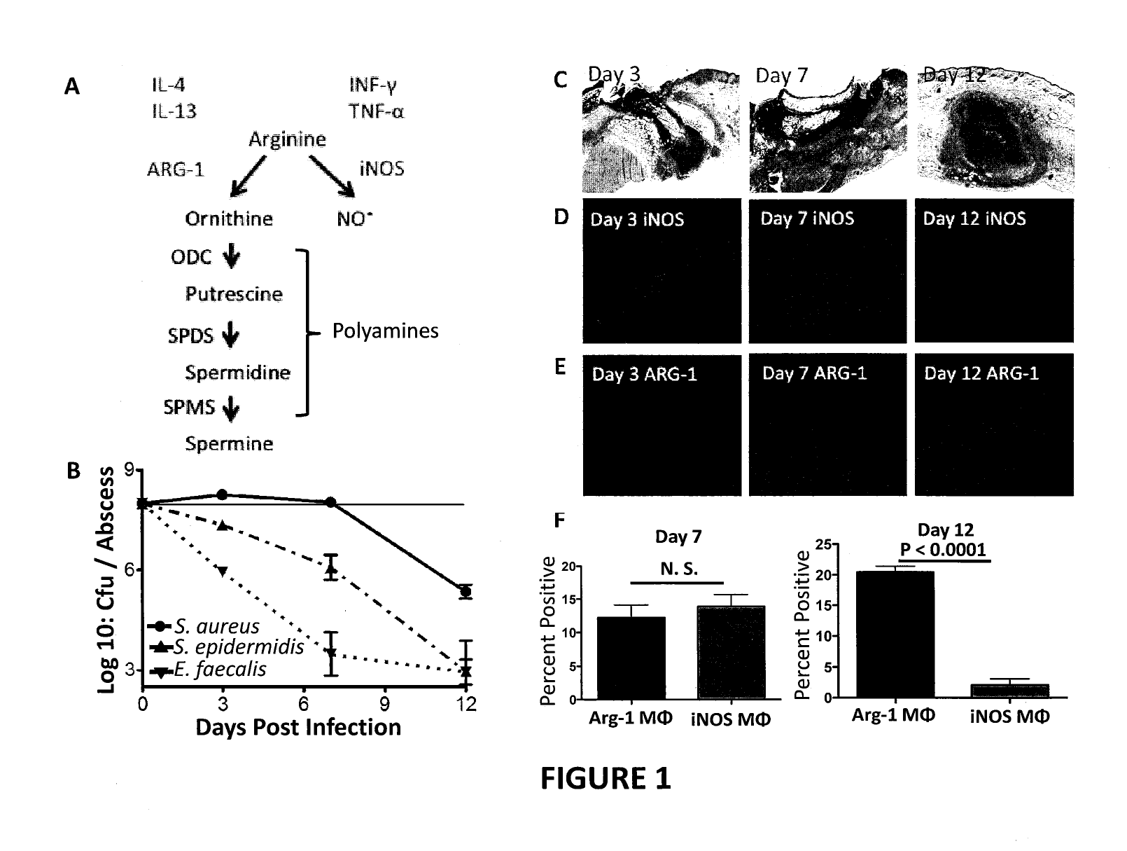

FIG. 2. Inhibition of polyamine synthesis exerts both direct and indirect effects on S. aureus killing. Panel A. The DFMO inhibits polyamine synthesis by blocking the activity of ornithine decarboxylase (ODC) and consequently eliminating M2-MΦs phenotypes. Polyamine resistance conferred to USA300 by SpeG significantly protects this strain from the toxic effects of host polyamines as seen by fewer viable bacteria at day 12 in wounds infected with ΔspeG. DFMO treatment eliminates the ΔspeG defect, but also drastically elevates the number if viable bacteria. Panel B. This observation is explained by the host response in DFMO treated animals. They exhibit no signs of the wound healing response at day 12 (no Arg, ODC or collagen expression, Red). Instead, they still exhibit signs of inflammation (iNOS Staining). Panel C. Similar to chemical inhibition of polyamine synthesis, genetic ablation of polyamine synthesis in the myeloid lineage specifically results in the lack of a wound healing transition and inability to resolve a MRSA SSTI.

FIG. 3. DFMO treatment limits PPAR-γ expression. Panel A. DFMO treated mice exhibit no PPAR-γ expression and consequently, no Arg-1 expression or wound healing. Panel B. Similarly, Arg-1 is not expressed in mice lacking PPAR-γ nor do these mice elicit a wound healing response. Panel C. Inhibition of polyamine synthesis limits PPAR-γ expression and M2-phenotypes in cultured macrophages stimulated with IL-4/10. Panel D. Viable bacteria within abscesses is greatly increased upon direct inhibition (GW9662), inhibition of expression (DFMO) or genetic elimination of PPAR-γ (PPAR-γ^{-/-}).

FIG. 4. Stimulating PPAR-γ hastens wound healing and S. aureus clearance. Panel A. Rosiglitazone and Pioglitazone both significantly reduced bacterial burdens over time. Panel B. Rosi-treated mice shift into the resolution phase earlier as seen by robust day 3 and 7 Arg-1 expression and collagen deposition. Panel C. Rosi-treatment in PPAR-γ^{-/-} mice has no effect eliminating the possibility of off-target Rosi effects. Panel D. MUFAs are likely reduced in PPAR-γ^{-/-} mice due to low SCD-1 expression.

FIG. 5. Rosiglitazone does NOT act by elevating polyamine levels that can kill most S. aureus. Panel A. WT polyamine-resistant USA300 (SF) survives within day 12 wounds better than its isogenic polyamine sensitive ΔspeG mutant. Rosiglitazone treatment affects both strains equally indicating that elevated polyamines are not solely responsible for the beneficial effects of Rosiglitazone. Panel B. Pathway for the synthesis of Spermine (Spm), Spermidine (Spd) and Putrescine (Put) from Arginine (Arg) and (Orn). Additionally, polyamines can be converted to Put by sequential reactions with Spm/Spd Acetyl Transferase (SSAT) and Polyamine Oxidase (PAO). Green shaded enzymes are encoded by gene directly activated by PPAR-γ. Pathways converge on Putrescine (Green Circle). Panel C. Total polyamine content of day 12 abscesses in mice untreated or treated with Rosiglitazone.

FIG. 6. PPAR-γ is NOT required for robust cationic antimicrobial peptide production by activated M1-MΦs. Previous reports have indicated a role for adipocyte PPAR-mediated antimicrobial peptide production that is critical for clearing S. aureus. We observe robust murine antimicrobial peptide production (CRAMP) in infected tissue during the inflammatory phase early on. CRAMP levels wane over the course of infection. Consistent with the inability of the PPAR-γ^{-/-} mice to transition from the inflammatory to the resolution phase, M1-MΦs (CD11b) from these animals still produce copious CRAMP even at day 12. Thus, the defect in clearing MRSA infections in PPAR-γ^{-/-} mice cannot be solely due to diminished CRAMP production by adipocytes as the bacteria are exposed to significant antimicrobial peptides from infiltrating M1-MΦs.

### DETAILED DESCRIPTION

The present invention will now be described more fully hereinafter. This invention may be embodied in different forms and should not be construed as limited to the embodiments set forth herein. Rather, these embodiments are provided so that this disclosure will be thorough and complete, and will fully convey the scope of the invention to those skilled in the art.

The terminology used in the description herein is for the purpose of describing particular embodiments only and is not intended to be limiting of the invention. As used in the description and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description herein is for the purpose of describing particular embodiments only and is not intended to be limiting of the invention.

All patents, patent applications and publications referred to herein are incorporated by reference in their entirety for the teachings relevant to the sentence and/or paragraph in which the reference is presented. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the embodiments of the invention described herein may be used in any combination. For example, features described in relation to one embodiment may also be applicable to and combinable with other embodiments and aspects of the invention.

Moreover, the embodiments of the present invention also contemplate that in some embodiments, any feature or combination of features set forth herein may be excluded or omitted. To illustrate, if the specification states that a complex comprises components A, B and C, in some embodiments, any of A, B or C, or a combination thereof, may be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value, such as, for example, an amount or concentration, is meant to encompass variations of ±20%, ±10%, +5%, ±1%, ±0.5%, or even +0.1% of the specified amount. A range provided herein for a measureable value may include any other range and/or individual value therein.

The present investigators have identified the stage of the host wound response that contributes most to the clearance of MRSA skin infections. The initial host response to infected wounds is highly inflammatory and is aimed at sterilizing the damaged tissue. Subsequently, the host shifts from the inflammatory phase into a resolution phase designed to repair damage and return normal tissue homeostasis. The post-inflammatory wound healing phase is associated with elevated production of polyamines and mono-unsaturated fatty acids (MUFAs) that are involved in the repair of epidermal tissue following infection. We have found that S. aureus is particularly susceptible to both polyamines and MUFAs, thereby explaining the rapid loss of live bacteria during the wound resolution phase, when these molecules are robustly synthesized. A key regulator known to be essential for maintaining a robust wound healing host response is the peroxisome proliferator-activated receptor gamma (PPARγ). This receptor responds to the accumulation of oxidized lipids that result from inflammation as well as several anti-inflammatory prostaglandins. We have shown that mice lacking PPARγ in immune cells (LysM-cre foxed PPARγ) cannot initiate the wound' healing phase and are unable to clear MRSA skin infections. Conversely, activation of PPARγ with anti-diabetic thiazolidinediones such as Rosiglitazone or Pioglitazone (but not Ciglitazone or Troglitazone) hasten the host wound healing response and significantly improved MRSA clearance. Additionally, these compounds had no effect in mice that lacked myeloid PPAR-γ. Accordingly, the present invention provides methods of using PPAR-γ agonists to treat Staphylococcus aureas infections.

In some embodiments, the staphylococcus infection is selected from the group consisting of Staphylococcus saprophyticus, Staphyloccocus xylosus, Staphyloccocus lugdunensis, Staphyloccocus schleiferi, Staphylococcus caprae, Staphylococcus epidermidis, Staphylococcus saprophyticus, Staphylococcus warneri, Staphylococcus aureus, Staphylococcus hominis, methicillin-resistant Staphylococcus aureus (MRSA), and Enterococcus faecalis, In particular embodiments, the staphylococcus infection is Staphylococcus aureus. In still other embodiments, the staphylococcus infection is methicillin-resistant Staphylococcus aureus (MRSA).

In particular embodiments, the PPAR-γ agonist is a thiazolidinedione. Thiazolodinediones or glitazones include a class of medications used in the treatment of diabetes. In some embodiments, the thiazolidinedione is selected from the group consisting of rosiglitazone, pioglitazone, netoglitazone, rivoglitazone, troglitazone and ciglitazone.

In some embodiments, the subject does not have type 1 diabetes or type 2 diabetes.

According to some embodiments of the present invention, the staphylococcus infection is present on the skin. “Skin,” as used herein, refers to any layer(s) of the skin including that on limbs, trunk, head, etc. Thus, the word “skin” is intended to include, but not be limited to, the epidermal and/or dermal layers, and may also include the underlying subcutaneous tissue. Mucosa (e.g., mouth, nasal, vaginal, etc.) and/or a surface of a subject's eye may also be treated.

In some embodiments, the staphylococcus infection is present in a wound. In some embodiments, the wound is a contaminated wound, infected wound or colonized wound.

In particular embodiments, the PPAR-γ agonist is administered orally, parenterally, by inhalation spray, topically, transdermally, rectally, nasally, sublingually, buccally, vaginally or via an implanted reservoir.

For oral administration, the PPAR-γ agonist of the present invention may be formulated into solid or liquid preparations such as, but not limited to, capsules, pills, tablets, troches, lozenges, chewing gum, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment of the present invention, a PPAR-γ agonist of the present invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They may provide a PPAR-γ agonist of the present invention in admixture with a dispersing or wetting agent, a suspending agent, and/or one or more preservatives. Suitable dispersing or wetting agents and suspending agents are exemplified by those already mentioned above. Additional excipients, for example, those sweetening, flavoring and coloring agents described above, may also be present.

A pharmaceutical composition including a PPAR-γ agonist of the present invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending a PPAR-γ agonist of the present invention in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

A PPAR-γ agonist of the present invention may also be administered parenterally, that is, subcutaneously, intravenously, intramuscularly, or interperitoneally, as injectable dosages of the PPAR-γ agonist in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which may be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

A parenteral composition of the present invention may contain from about 0.5% to about 90% or more by weight of a PPAR-γ agonist of the present invention in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

A topical formulation of the present invention may also include from about 0.5% to about 90% or more by weight of a PPAR-γ agonist of the present invention in a carrier. Exemplary topical formulations include, but are not limited to, a solution, an oil, an emulsion, a microemulsion, a suspension, an ointment, a lotion, a gel, a cream, a salve, a paste, a balm, a foam, a film, a patch and/or a suppository.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

A pharmaceutical composition of the present invention may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

In some embodiments, the PPAR-γ agonist is administered topically, intraperitoneally and/or subcutaneously.

In still other embodiments, the administration may be an immediate release administration or a sustained release administration.

The term “administering”, “administration”, and grammatical variants thereof, as used herein, refer to any mode of delivery to a subject. A PPAR-γ agonist of the present invention may be administered to a subject by any suitable route, including, but not limited to, orally (inclusive of administration via the oral cavity), parenterally, by inhalation spray, topically, transdermally, rectally, nasally, sublingually, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

The term “treating” and grammatical variants thereof, as used herein, refer to any type of treatment that imparts a benefit to a subject, including delaying, and/or reducing the progression of one or more symptom(s) and/or condition(s), reducing the severity of one or more symptom(s) and/or condition(s), etc. Those skilled in the art will appreciate that the benefit imparted by the treatment according to the methods of the present invention is not necessarily meant to imply cure or complete prevention.

“Prevent”, “prevention”, and grammatical variants thereof, as used herein, refer to avoiding the onset of a disease, disorder and/or a clinical symptom(s) in a subject relative to what would occur in the absence of the methods of the present invention and/or to avoiding an event in the life cycle of a microbial strain (e.g., colonization and/or proliferation) relative to what would occur in the absence of the methods of the present invention. In some embodiments, prevention is complete, resulting in the total absence of the disease, disorder and/or clinical symptom(s) (e.g., a total absence of growth of a pathogenic microbial strain). In some embodiments, prevention is partial, resulting in avoidance of some aspects of the disease, disorder and/or clinical symptom(s) (e.g., colonization by a pathogenic microbial strain but no subsequent proliferation).

The present invention finds use in both veterinary and medical applications. Suitable subjects or hosts of the present invention include, but are not limited to avians and mammals. The term “avian” as used herein includes, but is not limited to, chickens, ducks, geese, quail, turkeys, pheasants, ratites (e.g., ostrich), parrots, parakeets, macaws, cockatiels, canaries, finches, and birds in ovo. The term “mammal” as used herein includes, but is not limited to, primates (e.g., simians and humans), non-human primates (e.g., monkeys, baboons, chimpanzees, gorillas), bovines, ovines, caprines, ungulates, porcines, equines, felines, canines, lagomorphs, pinnipeds, rodents (e.g., rats, hamsters, and mice), and mammals in utero. In some embodiments of the present invention the subject is a mammal and in certain embodiments the subject is a human. Human subjects include both males and females of all ages including fetal, neonatal, infant, juvenile, adolescent, adult, and geriatric subjects as well as pregnant subjects.

In particular embodiments of the present invention, the subject is “in need of” the methods of the present invention, e.g., the subject has been exposed to a bacterial infection, it is believed that the subject will be exposed to a bacterial infection, and/or it is believed that the subject has been exposed to a bacterial infection. Such persons include, but are not limited to health care facility patients and/or personnel such as health care providers.

The administration step may be carried out prior to, during, and/or after exposure to a bacterial infection or a threat thereof Exemplary dosage regimens include, but are not limited to, once a day, twice a day, every other day, once a week, etc. for one or more day(s), week(s), month(s), and/or year(s). In particular embodiments of the present invention, the administering step is carried out to deliver an effective amount of a PPAR-γ agonist to treat or prevent MRSA. In some instances, the administering step is carried out by administering the PPAR-γ agonist of the present invention to a subject as a compound and/or included in a composition. In other instances, the administering step is carried out by administering the PPAR-γ agonist of the present invention to a subject as being integrated into or applied to a wound dressing or bandage applied to the skin or a wound. “Integrated” refers to being a part of the manufacturing process of the wound dressing or bandage.

As used herein, the term “effective amount” refers to an amount of a PPAR-γ agonist of the present invention that elicits a therapeutically useful response in a subject. Those skilled in the art will appreciate that the therapeutic effects need not be complete or curative, as long as some benefit is provided to the subject. In particular embodiments of the present invention, an effective amount of a PPAR-γ agonist of the present invention results in the detectable reduction of bacterial infection in a subject. Detection of bacteria may be accomplished by using methods and instruments known to those skilled in the art.

The present invention also provides methods of modulating a host wound response comprising administering an effective amount of a PPAR-γ agonist to host in an amount to elevate production of polyamines and/or mono-unsaturated fatty acids compared to the levels present in the absence of administration of a PPAR-γ agonist.

In some embodiments, an increase in the level of production of polyamines and/or mono-unsaturated fatty acids indicates repair of skin tissue. “Increase”, as used herein in refers to an elevation in activity or amount of at least about 5%, 10%, 25%, 50%, 75%, 100%, 150%, 200%, 300%, 400%, 500% or more.

In particular embodiments, the wound response is modulated during the inflammatory phase. In other embodiments, the wound response is modulated during the post-inflammatory phase.

In still other embodiments, the wound response is initiated by methicillin-resistant Staphylococcus aureus (MRSA).

In some embodiments, the PPAR-γ agonist is a thiazolidinedione. The thiazolidinedione may include rosiglitazone, pioglitazone, netoglitazone, rivoglitazone troglitazone and/or ciglitazone. In some embodiments, the thiazolidinedione may include rosiglitazone and/or pioglitazone.

The dosage regimen of the PPAR-γ agonist and/or composition including the same may be adjusted based on the exposure level and/or the subject. In some embodiments of the present invention, the amount of a PPAR-γ agonist of the present invention to be administered to a subject may vary according to considerations such as, but not limited to, the particular PPAR-γ agonist, the, dosage unit employed, the mode of administration, the period of treatment, the age and/or gender of the patient treated, and/or the nature and extent of the condition treated.

The present invention is explained in greater detail in the following non-limiting Examples.

### EXAMPLES

The foregoing is illustrative of the present invention, and is not to be construed as limiting thereof The invention is defined by the following claims, with equivalents of the claims to be included therein.

### Example 1

### Polyamine Production is Involved in Containing S. aureus SSTIs

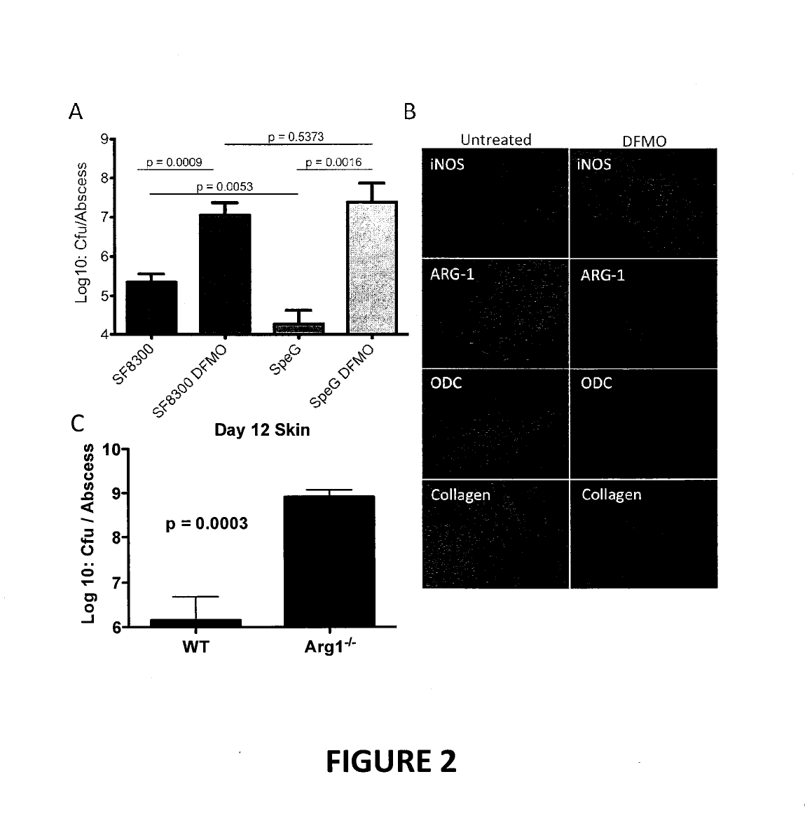

Inhibiting polyamine production chemotherapeutically with difluoromethylornithine (DFMO) diminished the normal accumulation of polyamines that occurs during the second week of infection (Thurlow et al. (2013) Cell Host Microbe 13, 100-107). This erases the advantage of polyamine resistant USA300 over polyamine-sensitive S. aureus (FIG. 2, Panel A). Additionally, DFMO treatment also prevented the host from entering the resolution phase at all. This is demonstrated by a lack of Arg-1 and ODC expression as well as limited collagen deposition at day 12 post inoculation (FIG. 2, Panel B). Thus, polyamine production is critical as these compounds directly kill sensitive S. aureus and are additionally involved in coordinating the transition to the wound resolution phase. Similarly, mice lacking Arg-1 specifically in the myeloid lineage exhibited defects in transitioning to the resolution phase. After 12 days of infection, the wounds were still highly inflamed with no sign of resolution phase markers. Consequently, the bacterial burdens were significantly higher than those of infected WT animals (FIG. 2, Panel C). Thus, chemical inhibition of global polyamine synthesis (DFMO) or genetic ablation of macrophage-specific polyamine synthesis (Arg-1^{-/-}) both result in a defect in the host transition into the pro-resolving healing phase that is critical for the clearance of S. aureus SSTIs.

### Example 2

### PPAR-γ is a Regulator That Modulates the Transition Into the Healing Phase and is Involved in the Clearance of MRSA SSTIs

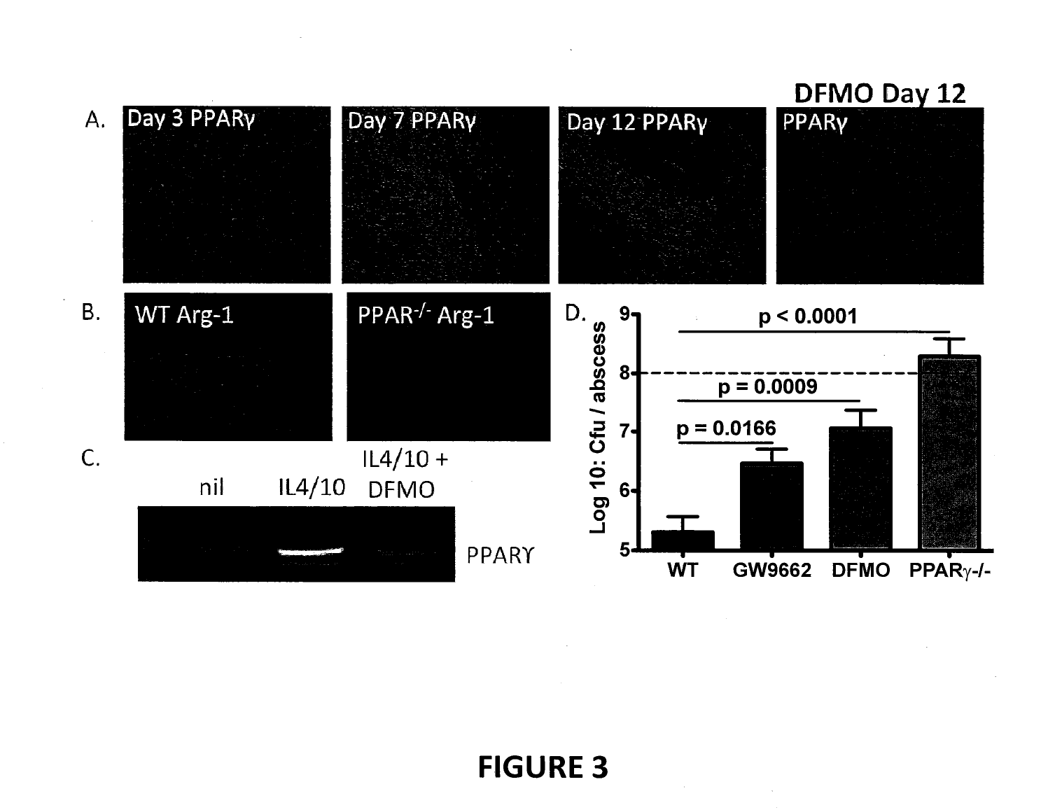

Peroxisome Proliferator Activator Receptor-γ (PPAR-γ) is a nuclear receptor that inhibits inflammation and redirects macrophages towards an M2 phenotype by driving Arg-1, Scd-1, SSAT and fatty acid synthesis gene expression. PPAR-γ is therefore involved in the development of adipocytes as well as M2-MΦs. In our murine SSTI model, PPAR-γ can be detected with the same kinetics as Arg-1, appearing on and after day 7 (FIG. 3, Panel A). Day 12 abscesses from mice lacking PPAR-γ in myeloid cells (PPAR-γ^{-/-}) are devoid of any Arg-1 signal (FIG. 3, Panel B). It is known that DFMO treatment can limit PPAR-γ expression in adipocytes (Uimari et ak, (2010) J. Cell Mol. Med. 14(6B), 1683-1692). Indeed, DFMO treatment also blocks PPAR-γ-expression in MΦs explaining the lack of resolution in DFMO treated mice (FIG. 3, Panels A and C). Additionally, Arg-1^{-/-} mice fail to express adequate PPAR-γ solidifying the role of polyamine synthesis in the expression and function of PPAR-γ. Furthermore, inhibiting the expression of PPAR-γ with DFMO, the activity of PPAR-γ with GW9662, a PPAR-γ inhibitor or genetic inactivation of PPAR-γ (PPAR-γ^{-/-}) all resulted in non-resolving MRSA SSTIs (FIG. 3, Panel D). Thus, polyamine synthesis is involved in the expression of PPAR-γ and the resulting transition into the critical wound-healing phase.

### Example 3

### PPAR-γ Activators Drive the Transition to Wound Healing and Promote Clearance of MRSA Infections

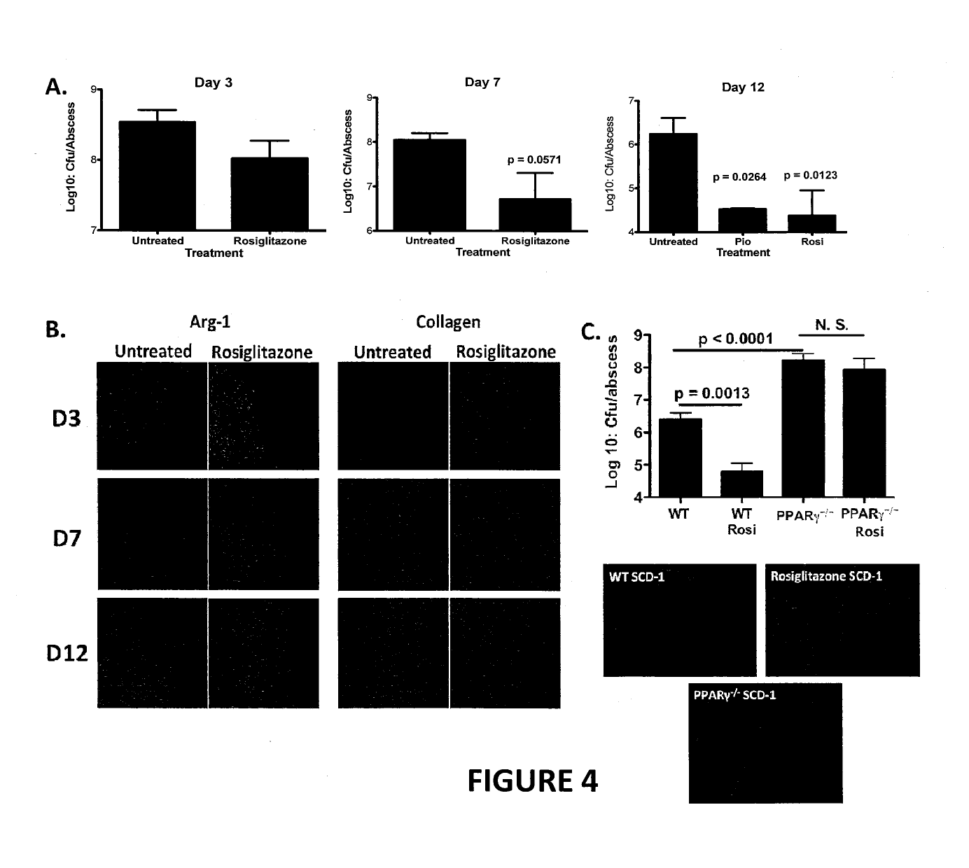

PPAR-γ agonists such as Rosiglitazone (Avandia) and Pioglitazone (Actos) dramatically shortened the duration of MRSA SSTIs (FIG. 4, Panel A). Treatment significantly reduced bacterial burdens at days 7 and 12, limited dissemination to other organs and reduced the average time to healing by ˜5 days. Other PPAR-γ agonists known to be less PPAR-γ-specific were not as effective (e.g. Traglitazone (Rezulin), data not shown). As predicted, staining tissue from MRSA skin abscesses in mice treated with Rosiglitazone (10 mg/kg i.p. injection daily) revealed wound healing signatures (e.g. Arg-1 expression and collagen deposition) as early as day 3 and peaking at day 7 (FIG. 4, Panel B). The beneficial effects of Rosiglitazone were absent in PPAR-γ^{-/-} mice indicating specificity with this treatment regimen. The mechanism of MRSA clearance hinges on the robust production by the host of mono-unsaturated fatty acids (MUFAs) that result from PPAR-γ stimulation. Indeed, Rosiglitazone-mediated activation of PPAR-γ drives fatty acid synthesis as well as Stearol Co-A Desaturase (SCD-1) the enzyme that converts saturated fatty acids into MUFAs (e.g. stearic acid into oleic acid). Accordingly, enhanced SCD-1 expression is seen in Rosiglitazone-treated wounds in a PPAR-γ-dependent fashion (FIG. 4, Panel D). Characterization of the fatty acid content of healing MRSA SSTIs after treatment with Rosiglitazone in WT, PPAR-γ^{-/-}, Arg-1^{-/-} and SCD-1^{-/-} mice, and whether Rosiglitazone treatment alters the fatty acid content, or merely enhances host production of anti-staphylococcal fatty acids, will be determined.

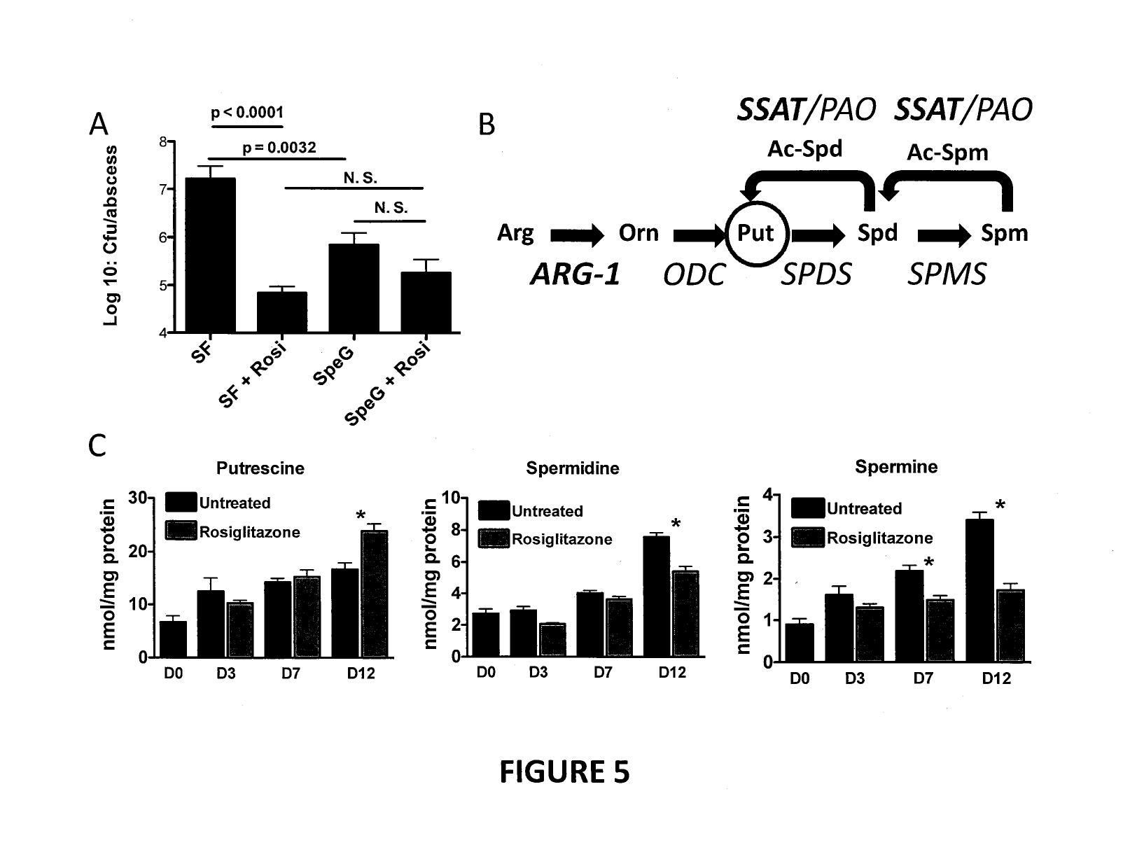

Polyamine-resistant and -sensitive MRSA strains are cleared equally by Rosiglitazone treatment (FIG. 5, Panel A). Second, PPAR-γ activates the expression of Arg-1 and SSAT, a gene pattern that would drive polyamine levels towards high putrescine, which is not toxic to MRSA (FIG. 5, Panel B). Indeed, this was determined by measuring tissue polyamines in mice treated with Rosiglitazone during MRSA skin infections (FIG. 5, Panel C). The normal increase in spermine/spermidine was absent in treated animals, rather they accumulated higher levels of putrescine, which is harmless to MRSA. It is believed that this pattern of polyamine levels benefits the fatty acid metabolism that is spurred by PPAR-γ.

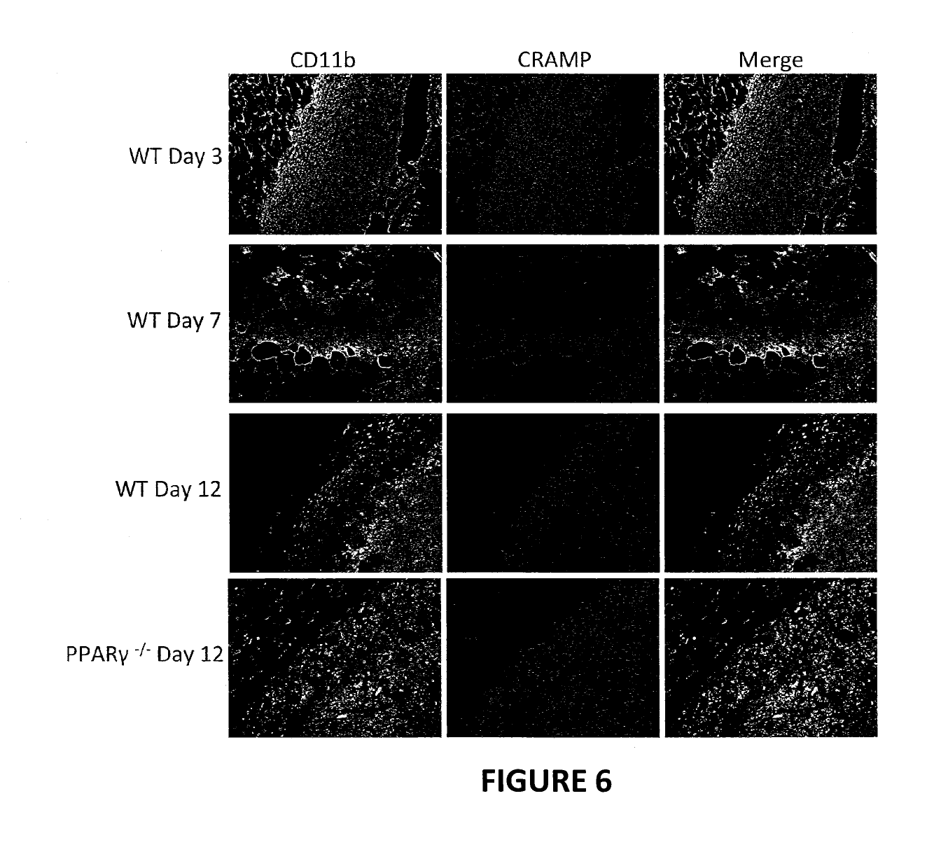

It has been concluded that adipocyte PPAR-γ is necessary for adipocyte-specific production of antimicrobial peptides such as CRAMP in mice (Zhang et al. (2015) Science 347(6217), 67-71). It was found that CRAMP production by macrophages (M1) far outweighed that of adipocytes and was most pronounced during the initial inflammatory phase of a MRSA SSTI (FIG. 6). Given the role of PPAR-γ in limiting the inflammatory phase, prolonged inflammation and elevated CRAMP levels in PPAR-γ^{-/-} mice was observed. Thus, the inability of PPAR-γ^{-/-} mice to clear MRSA infections could not be due to a lack of CRAMP expression. Thus, the PPAR-γ-dependent benefits of Rosiglitazone and Pioglitazone on MRSA SSTIs was not explained by enhanced CRAMP production. Rather elevated MUFAs upon PPAR-γ-stimulation are likely the mechanism of action.

The foregoing is illustrative of the present invention, and is not to be construed as limiting thereof. The invention is defined by the following claims, with equivalents of the claims to be included therein.

That which is claimed is:

1. A method of preventing or treating a Staphylococcus infection comprising administering an effective amount of a peroxisome proliferator-activated receptor (PPAR)-γ agonist to a subject in need thereof.

2. The method of claim 1, wherein the Staphylococcus infection is selected from the group consisting of Staphylococcus saprophyticus, Staphyloccocus xylosus, Staphyloccocus lugdunensis, Staphyloccocus schleiferi, Staphylococcus caprae, Staphylococcus epidermidis, Staphylococcus saprophyticus, Staphylococcus warneri, Staphylococcus aureus, Staphylococcus hominis, methicillin-resistant Staphylococcus aureus (MRSA), and Enterococcus faecalis.

3. The method of claim 1, wherein the Staphylococcus infection is Staphylococcus aureus.

4. The method of claim 1, wherein the Staphylococcus infection is methicillin-resistant Staphylococcus aureus (MRSA).

5. The method of claim 1, wherein the PPAR-γ agonist is a thiazolidinedione compound.

6. The method of claim 5, wherein the thiazolidinedione compound is selected from the group consisting of rosiglitazone, pioglitazone, netoglitazone, rivoglitazone, and troglitazone and ciglitazone.

7. The method of claim 1, wherein the Staphylococcus infection is present on the skin.

8. The method of claim 1, wherein the Staphylococcus infection is present in a wound.

9. The method of claim 8, wherein the wound is a contaminated wound, infected wound or colonized wound.

10. The method of claim 1, wherein the subject does not have diabetes.

11. The method of claim 1, wherein the PPAR-γ agonist is administered orally, parenterally, by inhalation spray, topically, transdermally, rectally, nasally, sublingually, buccally, vaginally or via an implanted reservoir.

12. The method of claim 11, wherein the PPAR-γ agonist is administered topically.

13. The method of claim 11, wherein the PPAR-γ agonist is administered intraperitoneally.

14. The method of claim 11, wherein the PPAR-γ agonist is administered subcutaneously.

15. A method of modulating a host wound response comprising administering an effective amount of a PPAR-γ agonist to a host in an amount to elevate production of polyamines and/or mono-unsaturated fatty acids compared to the levels present in the absence of administration of a PPAR-γ agonist.

16. The method of claim 15, wherein an increase in the level of production of polyamines and/or mono-unsaturated fatty acids indicates repair of skin tissue.

17. The method of claim 15, wherein the wound response is modulated during the inflammatory phase.

18. The method of claim 15, wherein the wound response is modulated during the post-inflammatory phase.

19. The method of claim 15, wherein the wound response is initiated by methicillin-resistant Staphylococcus aureus (MRSA).

20. The method of claim 15, wherein the PPAR-γ agonist is a thiazolidinedione.
